(19) United States
(12) Patent Application Publication
(10) Pub. No.: US 2022/0165354 A1
(43) Pub. Date: May 26, 2022

(54) METHODS, COMPUTER-READABLE MEDIA, AND SYSTEMS FOR ASSESSING WOUNDS AND CANDIDATE TREATMENTS

(71) Applicant: Drexel University, Philadelphia, PA (US)

(72) Inventor: Kara L. Spiller, Glenside, PA (US)

(73) Assignee: Drexel University, Philadelphia, PA (US)

(21) Appl. No.: 17/593,475

(22) PCT Filed: Mar. 16, 2020
(86) PCT No.: PCT/US2020/022905

§ 371 Date: Sep. 20, 2021

Related U.S. Application Data
(60) Provisional application No. 62/821,609, filed on Mar. 21, 2019.

(51) Int. Cl. G16B 25/10; C12Q 1/6883; C12N 15/10; G16B 40/20; G16H 50/20; G06N 20/10

(52) U.S. Cl. CPC G16B 25/10; C12Q 1/6883; C12N 15/1096; C12Q 2600/118; G16H 50/20; G06N 20/10; C12Q 2600/158; G16B 40/20

(57) ABSTRACT

One aspect of the invention provides a computer-implemented method of predicting whether a wound will heal or will not heal. The computer-implemented method includes: training a machine-learning algorithm utilizing at least: gene-expression values for at least m genes from a first clinical encounter for each of a plurality of training subjects; and a clinical diagnosis of a wound for each of the associated training subjects at a second, temporally later clinical encounter; and applying the previously trained machine-learning algorithm to gene-expression values for a corresponding set of m genes from a new subject having a wound; and presenting a prediction of whether the wound will heal generated by the previously trained artificial neural network machine-learning algorithm.

### CROSS-REFERENCE TO RELATED APPLICATION(S)

This application claims the benefit of priority of U.S. Provisional Patent Application Ser. No. 62/821,609, filed Mar. 21, 2019. The entire content of this application is hereby incorporated by reference herein.

### BACKGROUND OF THE INVENTION

Dysfunctional wound healing is a major complication of both type 1 and type 2 diabetes. Foot ulcerations, which occur in 15% of diabetic patients, lead to over 82,000 lower limb amputations annually in the United States, with a direct cost of $5 billion per year. The selection of an appropriate treatment strategy from dozens of choices available on the market, and knowing when to discontinue an ineffective treatment in favor of a different one, is critical to success. However, the process of wound healing is complex and difficult to assess. Currently, the gold standard of distinguishing between healing and non-healing is based on physician observation and wound size measurement. These methods are very subjective and prone to error, with only 58% positive predictive value.

### SUMMARY OF THE INVENTION

One aspect of the invention provides a computer-implemented method of predicting whether a wound will heal or will not heal. The computer-implemented method includes: training a machine-learning algorithm utilizing at least: gene-expression values for at least m genes from a first clinical encounter for each of a plurality of training subjects; and a clinical diagnosis of a wound for each of the associated training subjects at a second, temporally later clinical encounter; and applying the previously trained machine-learning algorithm to gene-expression values for a corresponding set of m genes from a new subject having a wound; and presenting a prediction of whether the wound will heal generated by the previously trained artificial neural network machine-learning algorithm.

This aspect of the invention can have a variety of embodiments. The machine learning algorithm can be an artificial neural network, a support vector machine, a binary classifier or series of binary classifiers or a decision tree.

In one embodiment, m is selected from the group consisting of 10, 50, 100, 500 and 1000.

The plurality of training subjects can include: a first subject group receiving a first wound treatment, and a second plurality of subjects receiving a second wound treatment.

The training step can further utilize gene expression values associated with the first and second wound treatment for the associated training subjects. The applying step can further provide a candidate wound treatment as an input to the previously trained machine-learning algorithm.

The method can further include proposing an optimum wound treatment for the new subject based on the gene expression values from the new subject. The gene expression values can be derived from a sample of debrided wound tissue.

The sample of debrided wound tissue can be collected at the first clinical encounter, stored in RNA-stabilizing solution and frozen until analysis.

The gene expression values can be derived by quantitative real-time polymerase chain reaction or by using a multiplex or high throughput gene expression analysis platform.

The wound can be a diabetic ulcer. The wound can be a diabetic foot ulcer, a diabetic ulcer of the leg, a venous ulcer or a pressure ulcer.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a schematic of the relationship between gene expression levels M1/M2 score and outcome.

FIG. 2 depicts a schematic of the relationship in FIG. 1 and predictions by a neural network.

FIG. 3 depicts a schematic overview of an embodiment of a method of the invention.

FIG. 4 depicts the scientific rationale behind tracking M1/M2 score using a model.

FIGS. 5A and 5B depict an evaluation of M1/M2 score as a biomarker for wound healing. FIG. 5A depicts M1/M2 score against time. FIG. 5B indicates that wound healing predictions based on M1/M2 are currently 90% accurate.

FIG. 6 depicts the scientific rationale behind macrophage-based machine learning algorithms.

FIG. 7A depicts a neural network-based machine learning algorithm constructed from the top 10 most highly expressed genes (listed on the left) selected from a panel of 227 macrophage phenotype-related genes, analyzed using NANOSTRING® technology. The network was trained on data collected from the first samples obtained from 13 patients and then tested on an additional 10 patients. This plot shows that the 10 genes were included in 9 hidden layers (H1 to H9) to predict one outcome (O1) at 12 weeks. The outcome contained three possible classifications: healing, remains open, or necessitates amputation.

FIG. 7B depicts prediction outcomes from the neural network of FIG. 7A. The neural network is currently 70% accurate (n=10 from multiple sites). The neural network correctly predicted 4/6 healing (67%) and 3/4 non-healing (75%).

FIGS. 8A and 8B depict the robustness and reliability of embodiments of the invention utilizing NANOSTRING® technology (FIG. 8A) and quantitative real-time polymerase chain reaction (qRTPCR) (FIG. 8B).

FIG. 9 depicts a comparison of macrophage-related biomarkers to wound size measurement.

FIG. 10 depicts levels of M1 and M2 biomarkers over time in in debrided wound tissue.

FIG. 11 depicts the in vitro cultivation of macrophages.

FIG. 12A depicts the conversion of gene expression data from in vitro-polarized macrophages into a combinatorial M1/M2a score (mean+/−SEM, n=5).

FIG. 12B depicts the change in M1/M2a score (relative to normal skin) over time in acute wounds (mean+/−SEM, n=3 pooled data from 15 samples), using data from Greco J A, et al., Burns. 2010; 36(2):192-204. Black asterisks indicate significance compared to normal skin.

FIG. 13 depicts the fold change in the M1/M2a score in healing (blue) and non-healing (red) DFUs over time relative to the first time point. Black asterisks indicate significance between healing and non-healing groups, while blue and red asterisks indicate differences over time within groups. Data are represented as mean+/−SEM, n=5 per group. *p<0.05, **p<0.01, ***p<0.001, ****p<0.0001.

FIG. 14 depicts higher M1/M2 scores in healing wounds.

FIG. 15A depicts a flowchart summarizing construction of a neural network built with in vitro samples.

FIG. 15B depicts a heat map of genes differentially expressed (DE) in M1 and M2 macrophages.

FIG. 15C depicts a graph of neural network (NN) performance as described by average predictive error based on the number of genes evaluated.

FIG. 16A depicts methods of polarizing primary human macrophages into four distinct phenotypes in vitro.

FIG. 16B depicts gene expression of a panel of common “M2” markers.

FIG. 16C depicts secretion of transforming growth factor beta-1 (TGFb1). the letter a indicates significance vs. other groups.

FIG. 17A depicts protein secretion by primary macrophages in vitro.

FIG. 17B depicts blood vessel formation by human endothelial cells and pericytes, with or without macrophages, in a 3D scaffold in vitro.

FIG. 18A depicts expression of genes related to ECM formation and degradation by M1, M2a, and M2c macrophages relative to unactivated (M0) macrophages.

FIG. 18B depicts stiffness, E, of matrices formed in vitro by human dermal fibroblasts cultured in the presence of conditioned media from macrophages.

FIG. 18C depicts images of the matrices quantified in FIG. 18B.

FIGS. 19A-19D depict a clustering analysis of M1, M2a, and M2c gene markers in normal human wound healing and application to DFUs. One cluster (FIG. 19A) consisted of genes that peaked in the early stages of healing, while another cluster (FIG. 19B) contained genes that peaked at later stages of wound healing. FIG. 19C depicts the composition of genes associated with each phenotype in the two clusters. FIG. 19D depicts the ratio of early stage M1 and M2c markers to late stage M2a markers in DFUs treated with the standard of care (n=12 samples from 7 responders, n=36 samples from 10 non-responders; *p<0.05). See Lurier E B et al., Transcriptome analysis of IL10-stimulated (M2c) macrophages by next generation sequencing. Immunobiology. 2017.

FIGS. 20A and 20B depict gene expression analysis of all 227 macrophage-related genes (FIG. 20A) and the top 10 most highly expressed genes (FIG. 20B) using the same sample analyzed in two different NANOSTRING® runs.

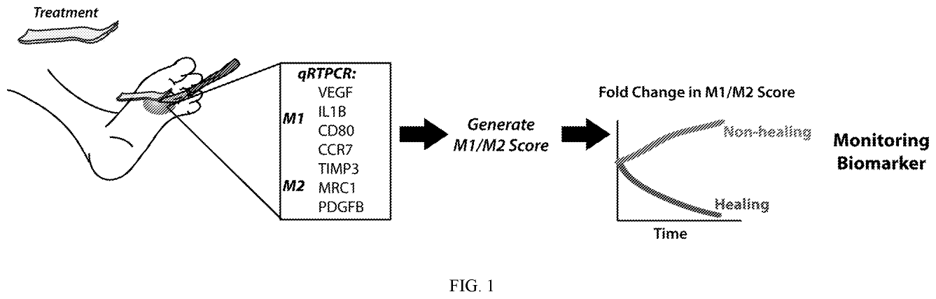

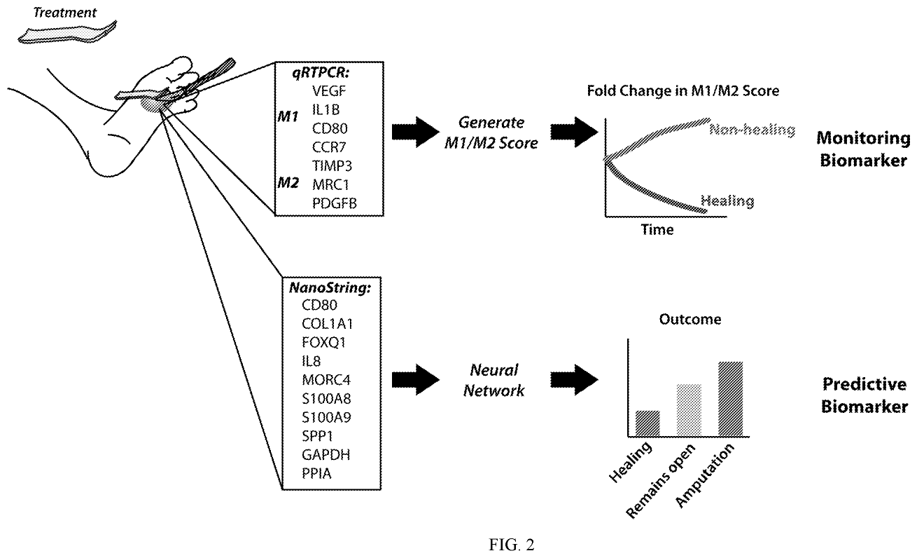

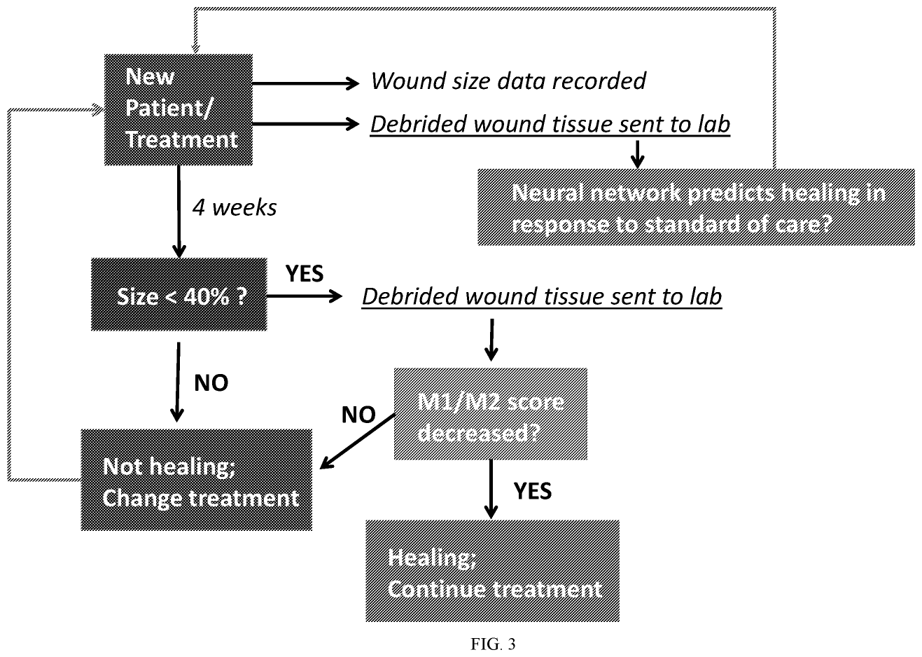

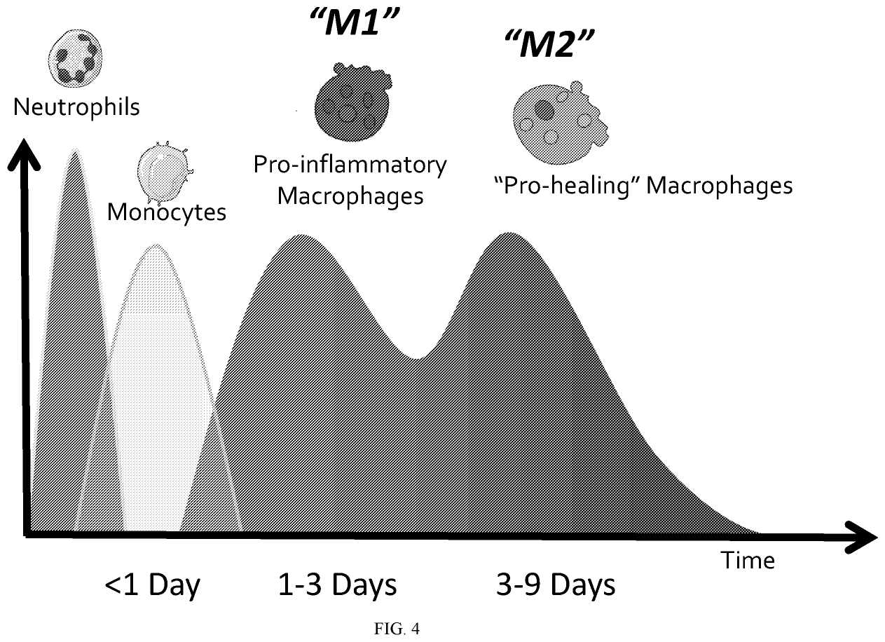

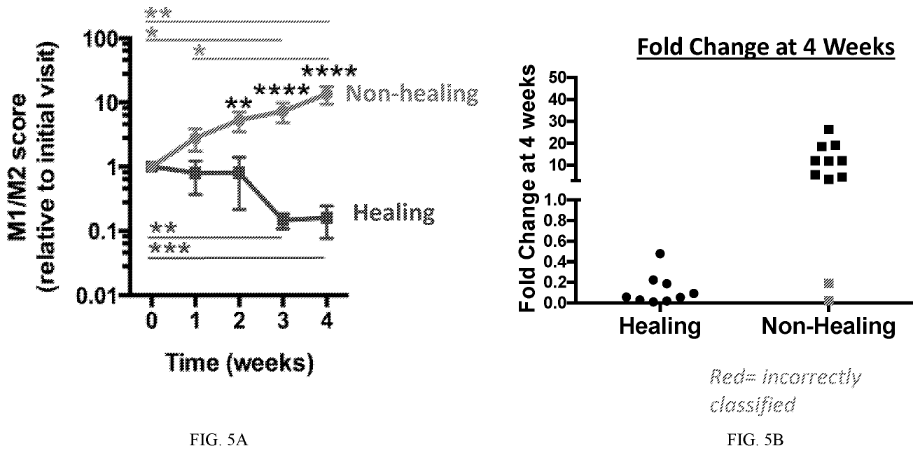

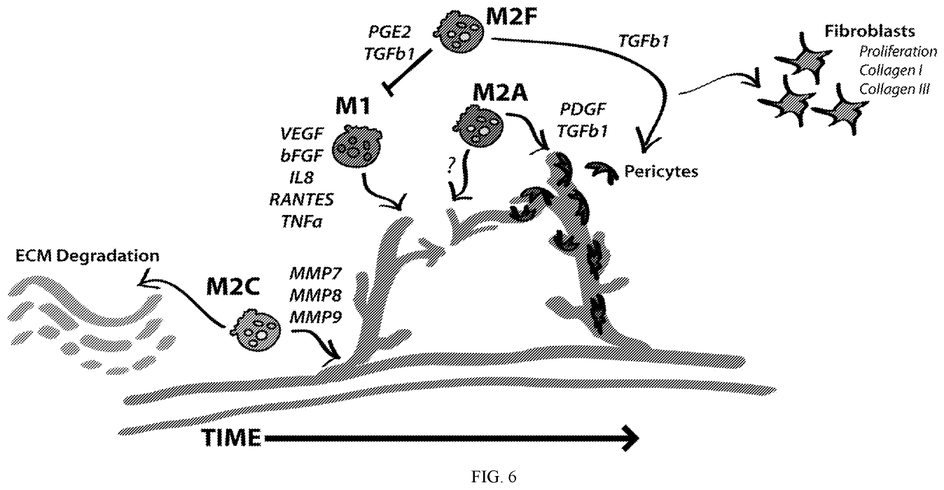

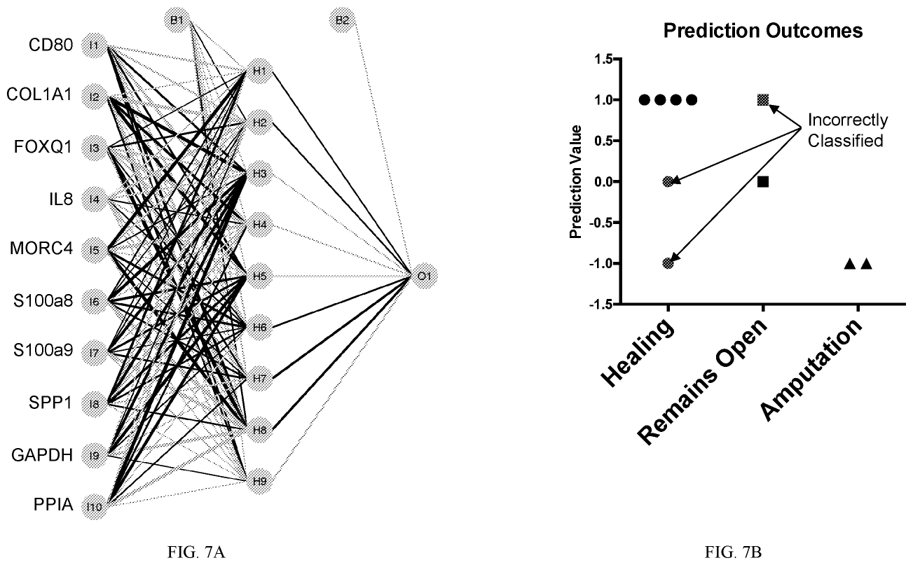

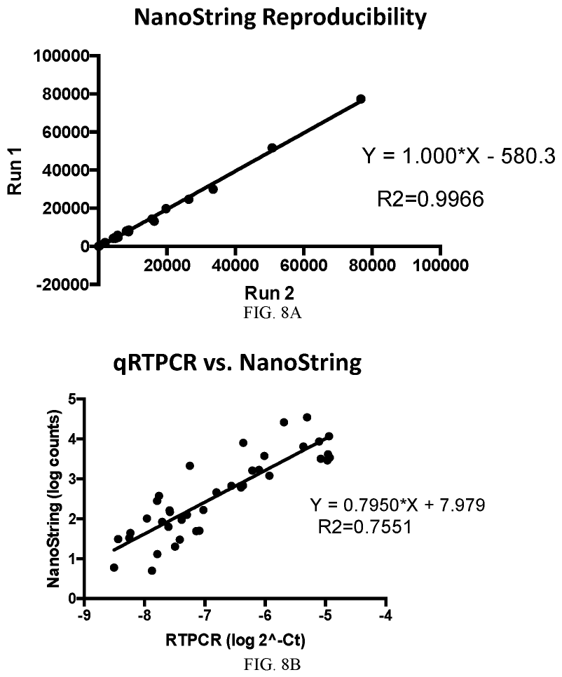

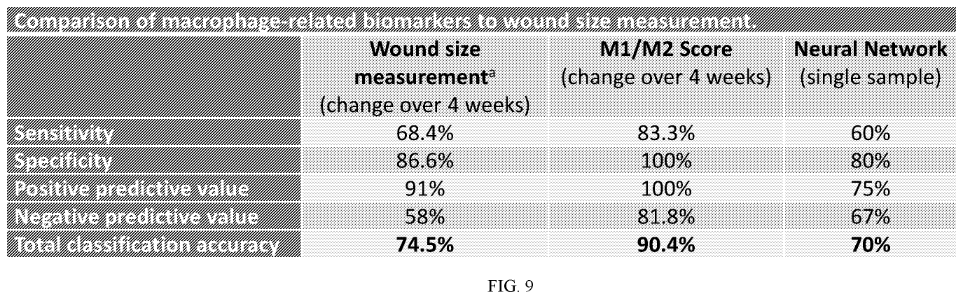

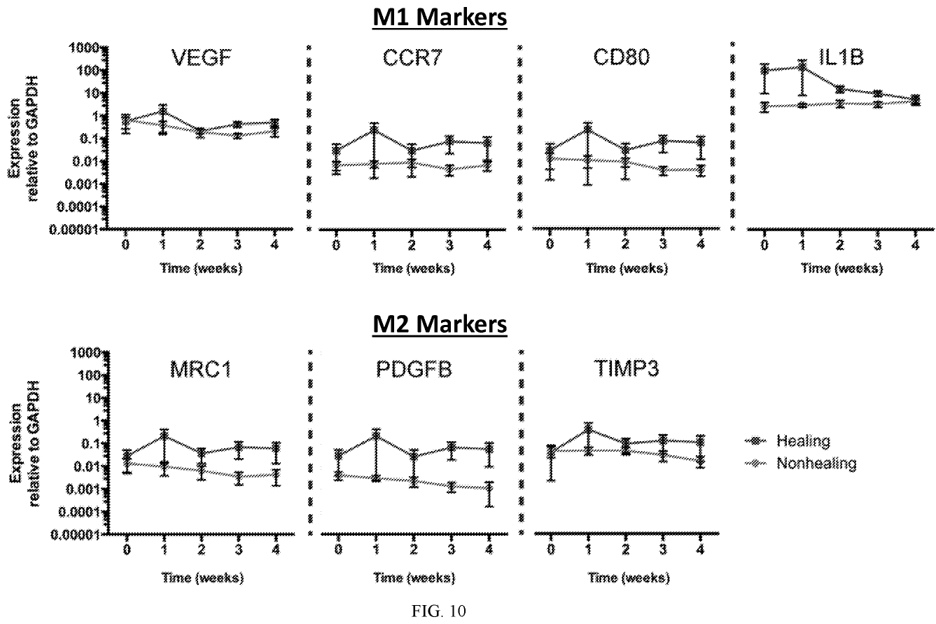

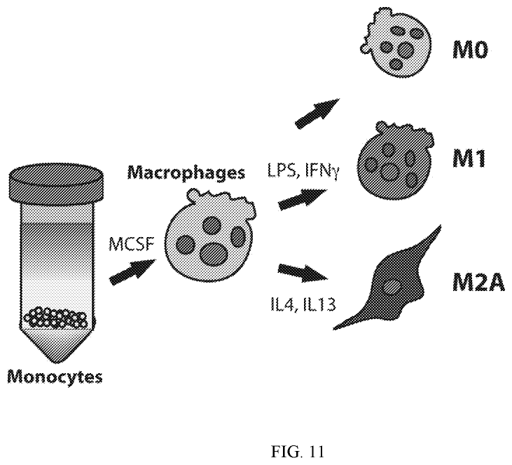

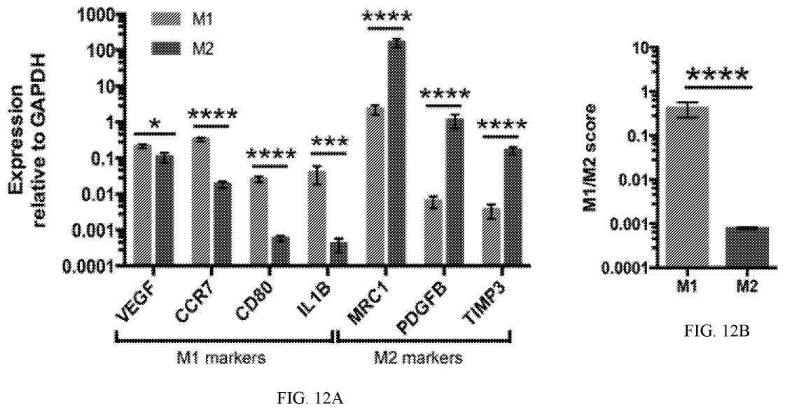

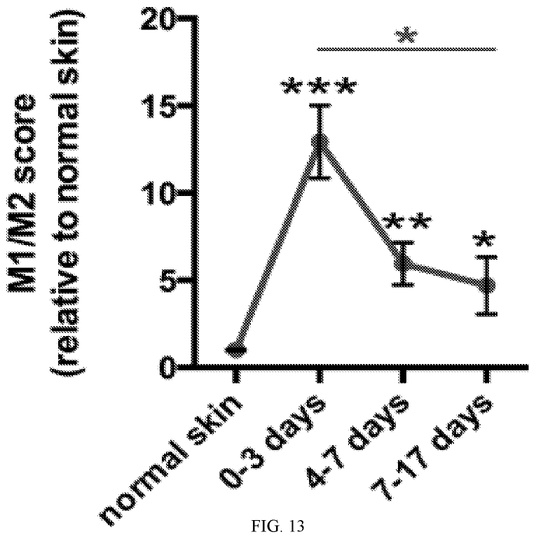

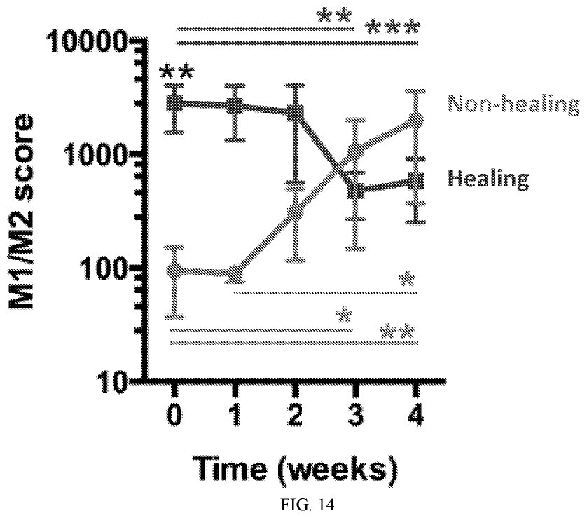

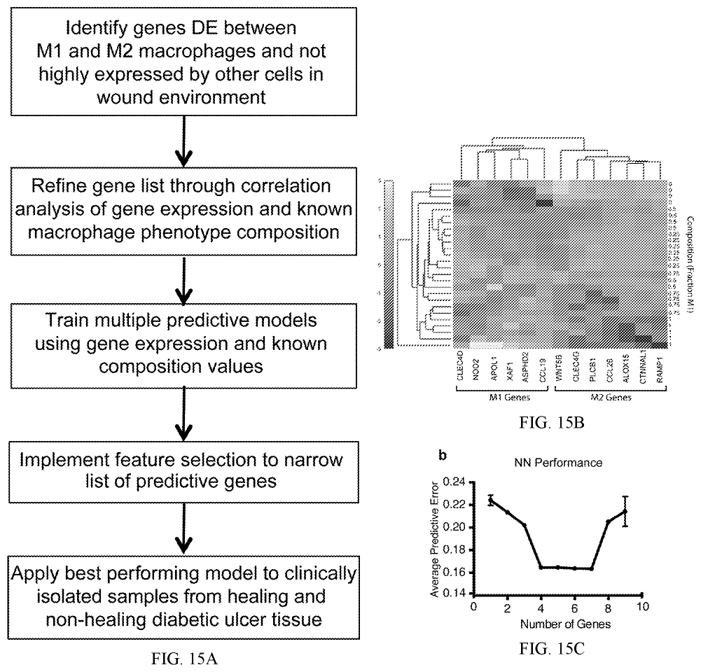

### DEFINITIONS

The instant invention is most clearly understood with reference to the following definitions:

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “ratio” refers to a relationship between two numbers (e.g., scores, summations, and the like). Although, ratios can be expressed in a particular order (e.g., a to b or a:b), one of ordinary skill in the art will recognize that the underlying relationship between the numbers can be expressed in any order without losing the significance of the underlying relationship, although observation and correlation of trends based on the ration may need to be reversed. For example, if the values of a over time are (4, 10) and the values of b over time are (2, 4), the ratio a:b will equal (2, 2.5), while the ratio b:a will be (0.5, 0.4). Although the values of a and b are the same in both ratios, the ratios a:b and b:a are inverse and increase and decrease, respectively, over the time period.

As used herein, the term “initial medical encounter” encompasses one or more related interactions with one or more medical professionals. For example, if a subject visits her primary care provider's office regarding a wound, her interactions with a medical assistant, nurse, physician's assistant, and/or physician would constitute a single “medical encounter.” Likewise, a subject's interactions with a plurality of medical professionals during an emergency department visit would also constitute an “initial medical encounter.” The term “initial medical encounter” also encompasses the first interaction with a medical professional specializing in wound care. For example, a subject's first appointment with a wound clinic would be considered an “initial medical encounter.” In regard to any particular medical issue, the initial medical encounter may be the first encounter in which the issue is addressed, regardless whether the subject has encountered the medical professionals previously. By way of non-limiting example, a patient may have a long history of interacting with a medical professional and the occasion on which a tissue sample is collected for analysis uses methods described herein will be the initial medical encounter with respect to this issue.

As used herein, RNALATER® refers to the specific formulation bearing that name and to RNA stabilizer solutions generally.

As used herein, the term “sample” includes biological samples of materials such as organs, tissues, cells, fluids, and the like. In one embodiment, the sample can be obtained from a wound. In other embodiments, the sample can be obtained from inflamed tissue such as tissue afflicted with Inflammatory Bowel Syndrome, Crohn's disease, and the like. In still another embodiment, the tissue can be cancerous tissue (in which an increase in M1/M2 ratio would be desired for inhibition of tumor progression). In still another embodiment, the sample can be obtained from an in vivo or in vitro testing platform such as a culture dish, a scaffold, an artificial organ, a laboratory animal, and the like.

As used herein, the term “wound” includes injuries in which the skin (particularly, the dermis) is torn, cut, or punctured. Examples of types of wounds that can be assessed using embodiments of the invention described herein include external wounds, internal wounds, clean wounds (e.g., those made in the course of a medical procedure such as surgery), contaminated wounds, infected wounds, colonized wounds, incisions, lacerations, abrasions, avulsions, puncture wounds, penetration wounds, gunshot wounds, and the like. Specific wound examples include diabetic ulcers, pressure ulcers (also known as decubitus ulcers or bedsores), chronic venous ulcers, burns, and medical implant insertion points. Embodiments of the invention are particularly useful in identifying non-healing wounds that are prevalent in diabetic and/or elderly subjects.

### DETAILED DESCRIPTION OF THE INVENTION

Previously proposed indicators of healing outcome biomarkers for diagnosis of non-healing wounds suffer from high variability between wounds, technical difficulties in detection methods, and impose burdens both on the patient and the care provider because the methods of detection are not a normal part of the wound care regimen. Furthermore, prior art methods provide little guidance as to what treatment methodology is most appropriate for a particular wound.

Aspects of the invention utilize genetic information about macrophage behavior and machine learning to identify differences between healing and non-healing in diabetic chronic wounds. Once trained using the gene expression values of a plurality of subjects undergoing a plurality of treatments, neural networks may then assist physicians by proposing a wound treatment for the new subject based on the gene expression values from the new subject. Macrophages are the central cell of the inflammatory response and are recognized as primary regulators of wound healing, with their phenotype orchestrating events specific to the stage of repair. Macrophages exist on a spectrum of phenotypes ranging from pro-inflammatory or “M1” to anti-inflammatory and pro-healing or “M2.” In early stages of wound healing (1-3 days), M1 macrophages secrete pro-inflammatory cytokines and clear the wound of debris. In later stages (4-7 days), macrophages switch to the M2 phenotype and promote extracellular matrix (ECM) synthesis, matrix remodeling, and tissue repair. If the M1-to-M2 transition is disrupted, depicted by persistent numbers of M1 macrophages, the wound suffers from chronic inflammation and impaired healing.

In one aspect, the invention provides a computer-implemented method of predicting whether a wound will heal or will not heal. The computer-implemented method includes: training a machine-learning algorithm utilizing at least: gene-expression values for at least m genes from a first clinical encounter for each of a plurality of training subjects; and a clinical diagnosis of a wound for each of the associated training subjects at a second, temporally later clinical encounter; and applying the previously trained artificial neural network machine-learning algorithm to gene-expression values for a corresponding set of m genes from a new subject having a wound; and presenting a prediction of whether the wound will heal generated by the previously trained machine-learning algorithm. In various embodiments, m is selected from the group consisting of 10, 50, 100, 500 and 1000. In various embodiments, training the machine learning algorithm further utilizes ratios of gene expression values.

In various embodiments, the plurality of training subjects comprises a first subject group receiving a first wound treatment, and a second subject group receiving a second wound treatment. A skilled person will appreciate that the first and second wound treatments may be any treatment applied to a wound as this is treated as another input in the training set for the neural network.

A skilled person will appreciate that a variety of machine learning algorithms are suitable for use in the methods of the invention and will be able to select an appropriate approach. Therefore, the specific choice of machine learning algorithm is not particularly limited. In various embodiments, the machine learning algorithm is an artificial neural network, a support vector machine, a binary classifier or series of binary classifiers or a decision tree.

In various embodiments, the training step further utilizes gene expression values associated with the first and second wound treatment for the associated training subjects; and the applying step further provides a candidate wound treatment as an input to the previously trained machine-learning algorithm. The gene expression values of subjects undergoing various wound treatments are provided to the neural network in the training set and associated with the wound treatment values in the sense that, without meaning to be limited by theory, the subject's cellular response to the wound treatment will drive the gene expression values. Having been trained with this data, in various embodiments the neural network is able to accept candidate wound treatment (e.g. a current or proposed wound treatment) and then predict the likely outcome of treatment. By way of non-limiting example, the trained neural network may predict using a subject's gene expression values and the current treatment as candidate wound treatment as inputs that if current treatment is maintained, then the wound will heal, will not heal or amputation will be required. In various embodiments, the method further includes proposing an optimum wound treatment for the new subject based on the gene expression values from the new subject. In various embodiments, the optimum wound treatment is the treatment that the neural network predicts will maximize the chance of wound healing. In various embodiments, the training step utilizes gene expression values associated with three, four, five or more wound treatments for associated training subjects.

A skilled person will recognize that the nature of the wound is not particularly limited as the neural network may be trained on gene expression values and wound treatments relating to a variety of wound types. In various embodiments, the wound is a diabetic ulcer. In various embodiments the wound is a diabetic foot ulcer, a diabetic ulcer of the leg, a venous ulcer or a pressure ulcer. Likewise, the neural network can be further trained using subject demographics or medical data.

In various embodiments, the gene expression values are derived from a sample of debrided wound tissue. In various embodiments, the sample of debrided tissue is collected at the first clinical encounter, stored in RNA-stabilizing solution and frozen until analysis. In another embodiment, surgical debridement can be performed using various surgical tools such as a scalpel, a laser, and the like. Advantageously, harvesting of debrided tissue avoids the challenges associated with more invasive approaches such as using punch biopsies while providing sufficient quantities of human wound tissues for quantitative analyses of the cellular content using tissue that would otherwise be discarded. Although relatively non-invasive procedures can be used, the samples used herein can also be obtained through invasive procedures such as punch biopsies, shave biopsies, incisional biopsies, excisional biopsies, curettage biopsies, saucerization biopsies, fine needle aspiration, and the like.

In some embodiments, the sample is obtained during an initial medical encounter. In other embodiments, the sample (which may be a first sample or a subsequent sample) is obtained during a subsequent medical encounter. For example, it may be desirable to treat an infection, correct vascular insufficiency, and/or address other conditions before debriding the wound and obtaining the sample. In some embodiments, the medical professional will obtain the sample after determining that wound healing in response to current treatment is unsatisfactory and other treatment options should be considered.

A skilled person will further recognize that a variety of methods of measuring gene expression may be employed. In various embodiments, the gene expression values are derived by quantitative real-time polymerase chain reaction or by using a multiplex or high throughput gene expression analysis platform. In various embodiments, the high throughput gene expression analysis platform may be a micro array. In various embodiments, the microarray may be a NANOSTRING® NCOUNTER® system.

### Examples

### Materials and Methods

Description of the collection and transport of biosamples. Instead of throwing away the tissue that is routinely removed during debridement, it will be collected and analyzed for biomarker. In various embodiments of the methods of the invention two samples are collected per DFU: a first debridement is performed to remove the necrotic tissue that typically covers the wound. This sample is stored for subsequent microbiome analysis (not included in the present proposal). Then, a second debridement is performed into the viable tissue within the DFU. Tissue samples will be stored in a small vial containing RNALATER® stabilization solution (ThermoFisher) and shipped overnight at room temperature to the laboratory for analysis. RNALATER® stabilization solution is nontoxic and non-noxious, and comes in pre-filled vials, making it an ideal sample collection system for the clinical setting. In addition, several published studies have shown that this method maintains stability of RNA in tissues for up to 7 days at room temperature, up to 30 days at 4° C., and indefinitely at −20° C. or colder. Upon receipt, samples will be immediately transferred to −80° C. for long-term storage.

### Protocol for the Biomarker Measurement:

RNA extraction—Samples will be thawed, removed from RNALATER® stabilization solution and homogenized in TRIZOL® solution (ThermoFisher) in individual vials using a bead beater. RNA will be extracted using chloroform and subsequently purified using the RNEASY® Micro Kit (Qiagen), according to routine methods. RNA quality and concentration will be measured using a BIOANALYZER® machine (Agilent Technologies).

M1/M2a score—RNA is converted to cDNA using the High Capacity cDNA synthesis kit (ThermoFisher) and gene expression is measured using SYBR® Green reagents (ThermoFisher) and 20 ng RNA per reaction, according to standard practice and previously published methods. The M1/M2a score is calculated by taking the linear sum of the expression of 4 M1 markers (IL1b, CCR7, CD80, VEGF) divided by the sum of 3 M2a markers (MRC1, TIMP3, PDGFB). This score is then tracked over 4 weeks for each patient. A decrease in the score (or fold change less than 1) is used to classify healing at 12 weeks, while an increase in the score (or fold change greater than 1) is used to classify non-healing (either amputation or remaining open at 12 weeks).

Neural network—NANOSTRING® technology will be used to measure expression of the 10 genes identified in the pilot study (CD80, COL1A1, FOXQ1, IL8, MORC4, S100a8, S100a9, SPP1, GAPDH, and PPIA), as well as 8 external RNA control consortium (ERCC) positive control and 8 negative control transcripts, using 100 ng of RNA total per sample, according to the manufacturer's recommendations. The raw counts (normalized to positive and negative controls) are used as input into the previously developed neural network, which reports an output of healing outcome as one of three possible outcomes: healing, remains open, or necessitates amputation. In addition, because this is a multiplex method, more genes can be added without affecting the cost (for the purposes of this study up to 13 more genes can be added without affecting the cost at all). For this reason the 7 genes that comprise the M1/M2a score are also measured, which may facilitate scale-up of that biomarker, as well as 6 genes identified in other studies.

Data on the reliability of the measurement. Both qRTPCR and NANOSTRING® technology are considered highly reliable, reproducible techniques for gene expression analysis, even for highly degraded samples. Quality control studies have shown very little technical variation in NANOSTRING® analysis of DFU samples, as evidenced by a Spearman's correlation coefficient of 0.98 (p<0.0001) when analyzed across 227 macrophage phenotype-related genes and 0.955 (p<0.0001) using only those 10 genes included in the neural network (FIGS. 22A and 22B). To further validate the reliability of the measurement, some samples in each batch of analysis will be repeated on other batches, and reference samples of in vitro-polarized macrophages will also be included in each run to confirm that differences are due to actual differences and not technical variability.

### Clinical Study Design.

Patient enrollment and clinical data collection. Inclusion criteria include diagnosis of diabetes, having one open DFU that has not healed for at least 8 weeks at the time of enrollment (i.e. chronic status), ankle brachial index between 0.75-1.2, and no signs of osteomyelitis or infection probing to the bone or tendon. Samples collected from both males and females will be analyzed. Subjects should be treated as usual according to the best judgment of their clinician, which includes weekly or biweekly debridement with a scalpel, offloading, and treatment with moist wound dressings (including moist gauze as well as neutral collagen-based materials). Subjects should not be treated with amniotic membrane-derived materials, which have been found to be anti-inflammatory to macrophages, and thus may affect the predictive capability of the neural network because it was designed to predict non-responsiveness to the standard treatment. With respect to clinical information to collect from each subject, medical data that may become useful at the analysis stage include: site of the ulcer, ulcer surface area, depth, and treatment; age, self-identified gender, smoking status, weight/body mass index (BMI), hemoglobin A1c levels, glucose levels, other comorbidities (e.g. renal failure, cardiac disease, hypertension, etc.), whether they are taking insulin or other drugs and the duration of treatment. Information about the specific treatment employed is collected.

Sample collection and storage. At each treatment over the course of 4 weeks, instead of throwing away the viable tissue that is removed during debridement, it is stored in a small vial containing RNALATER® stabilization solution (ThermoFisher) and shipped overnight to the laboratory for analysis. Importantly, this procedure has been validated by determining that this method of sample collection, storage and shipping yields sufficient RNA samples for analysis by qRTPCR and NANOSTRING® technology. Upon receipt, samples are immediately transferred to −80° C. for long-term storage.

Power analysis. From pilot studies of the M1/M2a score, a sensitivity of 83% and a specificity of 100% has been calculated. Even if the actual specificity of the validated test is lower than 100% but higher than the sensitivity, then the required number of subjects for validation is limited by the test's sensitivity. Using a conservative estimate of an actual sensitivity of 80%, non-healing ulcer prevalence of 47%, 131 subjects are required to calculate a 95% confidence interval with a width of 0.1 in order to validate this test. The pilot study of the neural network resulted in a sensitivity of 60% and specificity of 80%. Calculating the required number of subjects based on this sensitivity at the same confidence intervals as described above yields a requirement of 197 subjects. These calculations are based on the methods described in Buderer N M. Statistical methodology: I. Incorporating the prevalence of disease into the sample size calculation for sensitivity and specificity, Acad. Emerg. Med. 1996; 3(9):895-900. Ideally the neural network would be further trained on an additional 25% of the subjects (which is expected to improve its accuracy) and then tested on the 197 required subjects, bringing the total number of subjects required to 250. Because the M1/M2a score and the neural network assays can be run on samples from the same subjects, the total number of required subjects to validate both tests is therefore 250.

Primary data analysis. The M1/M2a score is calculated for each sample collected weekly or biweekly over 4 weeks of treatment, as in pilot studies, by dividing the linear sum of the expression levels (2\hat{ }-Ct) of the four M1-associated genes (CCR7, IL1B, VEGF, CD80) by the linear sum of the expression levels of the three M2a-associated genes (MRC1, PDGFB, TIMP3), measured using qRTPCR. After 12 weeks from enrollment, subjects are classified as healing or non-healing based on whether their wound fully closed by this time point. Pilot studies used a cutoff in the fold change of the M1/M2a score of 1, so that higher than 1 indicates an increase and non-healing, while lower than 1 indicates a decrease and healing. ROC curves are constructed using sensitivity and specificity calculated when varying the threshold around 1 to determine the optimal cutoff point.

A previously developed neural network model is used to predict the 12-week outcome using NANOSTRING® analysis of the first sample collected for each patient as in the preliminary studies, using 10 genes (CD80, COL1A1, FOXQ1, IL8, MORC4, S100a8, S100a9, SPP1, GAPDH, and PPIA), and 9 hidden layers to predict outcome as complete wound closure, remains open, or necessitates amputation (keeping in mind that the decision to amputate is determined according to the best judgment of the clinician in consultation with their patient). Classification accuracy will be determined for each of the three outcomes, and sensitivity and specificity will be calculated by dichotomizing the three outcomes into healing vs. non-healing.

Secondary analysis. In addition to validating the previously developed biomarkers, any clinical factors that affect them are determined, which is useful for improving understanding of DFU healing and may also improve the predictive capabilities of the proposed biomarkers. For example, ulcer area, hemoglobin A1c levels, body mass index (BMI), smoking status (in terms of packs per day), age, and gender have all been reported to affect wound healing outcome, but their effects on the wound tissue locally are not known. Multivariable regression is performed in R to determine the effects of clinical factors on the M1/M2a score as a continuous variable. These factors are included as layers in the neural network machine learning algorithm to determine the effects on its predictive ability. Finally, an additional 13 genes can be included in this study, since up to 23 genes can be included in NANOSTRING® analysis for the same price as 10 genes. Therefore 6 additional genes from the M1/M2a score may be added (note that CD80 appears in both biomarker panels), which would allow the M1/M2a score to be measured using either qRTPCR or NANOSTRING® technology, and/or genes related to other biomarkers of interest may be included.

RNA Extraction, Complementary DNA Synthesis, and qRT-PCR

Wound samples were thawed at room temperature and processed for RNA extraction using TRIZOL® Plus RNA purification kit according to the manufacturer's instructions. Extracted RNA was eluted in 30 uL of RNAse-free water and stored at −80° C. until synthesis of complementary DNA (cDNA) using the APPLIED BIOSYSTEMS® High-Capacity cDNA Reverse Transcription Kit available from Life Technologies. Lastly, quantitative analysis of expression of multiple markers of macrophage phenotype was performed using qRT-PCR with GAPDH as a reference gene, as previously described in K. L. Spiller et al., “The role of macrophage phenotype in vascularization of tissue engineering scaffolds,” 35(15) Biomaterials 4477-88 (May 2014) (hereinafter “Spiller”).

The results of the Experimental Examples are here described.

Wound healing prediction based on gene expression values Currently, complete wound closure is the only accurate and objective indicator of treatment efficacy, and this can take several months or even years. As a result, many promising therapies are not approved because they fail to achieve closure within the predetermined time frame (usually 12 or 20 weeks). The only accepted surrogate endpoint is a change in wound size, in which a 40-50% reduction over 4 weeks is used to indicate healing at 12 weeks. While this method generally performs well at predicting those ulcers that will not heal (91% positive predictive value), it drastically underperforms at predicting those that are healing (58% negative predictive value). As a result, many wounds are not treated aggressively because they are incorrectly classified as healing. Only months later do clinicians realize that they should have switched treatments earlier, which increases costs and makes a healing outcome less and less likely. In addition, the wound size measurement method conveys only superficial wound characteristics. Thus, it is extremely difficult to determine why some patients respond to treatment while others do not, and why some wound care products are effective while others are not. Interestingly, many wound care companies evaluate new products in clinical trials in which a run-in period is used to remove “healers” from the study using a cut-off of 25% reduction in wound size over 2 weeks; because this method is not accurate, the products are not tested on patients who may benefit from the treatment and they are tested on patients who would have otherwise healed in response to the standard of care.

In contrast, the methods of the invention in embodiments comprising measuring the change in the M1/M2a score derived from gene expression in debrided wound tissue outperforms wound size measurement in terms of sensitivity, specificity, positive predictive value, negative predictive value, and overall classification accuracy (Table 1). While the neural network machine learning algorithm is not yet as accurate as the other methods in terms of overall classification accuracy, its negative predictive value is already better than wound size measurement, and its accuracy is expected to improve as the training data set becomes larger and more diverse and as clinical factors such as age and smoking status are incorporated into it. Most excitingly, it works with just a single sample obtained at the patient's first visit. Finally, it predicts one of three possible outcomes by 12 weeks: complete wound closure, remains open, or necessitates amputation.

TABLE 1
Comparison of macrophage-related
biomarkers to wound size measurement.

| | Wound size | | Neural |
|---|---|---|---|
| | measurement^{a} | M1/M2a Score^{b} | Network^{c} |
| | (change over 4 | (change over 4 | (single |
| | weeks) | weeks) | sample) |
| Sensitivity | 68.4% | 83.3% | 60% |
| Specificity | 86.6% | 100% | 80% |
| Positive predictive value | 91% | 100% | 75% |
| Negative predictive value | 58% | 81.8% | 67% |
| Total classification | 74.5% | 90.4% | 70% |
| accuracy | | | |

^{a}Based on a study of 203 patients, in which a 53% change in wound size over 4 weeks was used to classify the outcome 12 weeks.
^{b}Based on 3 studies of a total of 21 patients, in which a decrease in the score over 4 weeks classified the outcome at 12 weeks as healing while an increase classified non-healing (remains open or requires amputation) (Nassiri, Bajpai).
^{c}Based on a study involving 13 patients in the training set and 10 patients in the validation set, in which analysis of a single sample obtained at the first visit was used to classify outcome at 12 weeks (not yet published).

Diverse macrophage phenotypes. In normal wound healing, monocytes are recruited from the circulation to the site of injury, where they differentiate into macrophages, release inflammatory cytokines and recruit other immune cells. In early stages of wound healing (1-3 days), macrophages exhibit a predominantly pro-inflammatory phenotype, also referred to as “M1,” which initiates the process of healing. In later stages (4-7 days), macrophages switch to an “alternatively activated” or “M2” phenotype. M2 macrophages promote extracellular matrix (ECM) synthesis and remodeling and resolution of the healing process. If the M1-to-M2 transition is disrupted, depicted by persistent numbers of M1 macrophages, the wound suffers from chronic inflammation and impaired healing. It is not well understood why diabetic wound macrophages are stalled in the M1 state; probable causes include defective clearance of apoptotic cells, hyperclemia, hypoxia, altered nutrient utilization and metabolism, chronic infection, and likely many more.

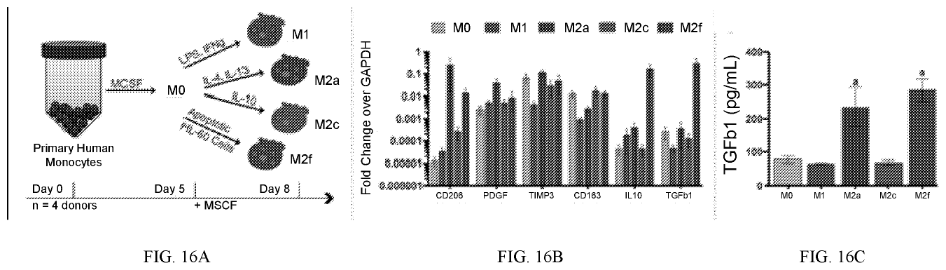

While it is known that the M1-to-M2 transition is critical for successful wound healing, the mechanisms behind their diverse behaviors are poorly understood. The basic mechanisms by which macrophages of different phenotypes influence angiogenesis, fibroblast migration, and ECM deposition are under investigation, which directly lead into efforts to design better diagnostics and treatments for DFU care. M1 macrophages are generated in vitro using the pro-inflammatory stimuli interferon-gamma (IFNg) and lipopolysaccharide (LPS), while M2a macrophages are generated through the addition of the Th2 cytokines interleukin-4 (IL4) and IL13 (FIG. 16A). In addition, two more distinct phenotypes of macrophages that play critical roles have been characterized in various stages of wound healing. Using next generation sequencing (RNAseq), it was found that M2c macrophages, which are stimulated with IL10, secrete high levels of critical proteins involved in ECM remodeling, such as matrix metalloprotease-7 (MMP7), MMP8, and MMP9. Another distinct phenotype results from the phagocytosis of apoptotic neutrophils, in the process called efferocytosis. This phenotype (herein, “M2f”) is characterized by increased production of anti-inflammatory cytokines like IL10, transforming growth factor-β1 (TGFB1) (FIGS. 16B, 16C), and prostaglandin-E2 (PGE2), allowing it to inhibit the inflammatory actions of M1 macrophages. Importantly, efferocytosis is known to be defective in diabetic wounds, further highlighting the significance of this phenotype.

Using these carefully defined polarized macrophages in vitro, it was found that human M1 macrophages secrete the potent pro-angiogenic growth factor vascular endothelial cell growth factor-A (VEGFA), which is critical for initiation of angiogenesis, while M2a secrete the late stage blood vessel-stabilizing factor platelet-derived growth factor-BB (PDGFBB), in keeping with the sequential actions of these phenotypes in wound healing (FIG. 17A). Indeed, the sequential addition of human M1 and M2a macrophages to a 3D model of vascularization in vitro enhanced blood vessel formation compared to the addition of either phenotype alone (FIG. 17B). These results are in keeping with studies that have shown that VEGF-secreting inflammatory macrophages are critical for the initiation of tissue vascularization in murine skin wounds, while IL4 receptor signaling (which promotes M2a polarization) in macrophages is important for vascular maturity and stability. Similarly, it has been found that M2a macrophages express the highest levels of genes associated with ECM formation, while M2c macrophages express the highest levels of genes associated with ECM degradation and remodeling (FIG. 18A). Moreover, M2a macrophages stimulate human dermal fibroblasts to produce the stiffest matrices in vitro (FIGS. 18B and 18C), further supporting their role in later stages of wound healing. Collectively, these studies suggest a sequential and synergistic role of M1 and M2a macrophages in various aspects of wound healing, and inspired development of the M1/M2a score described herein.

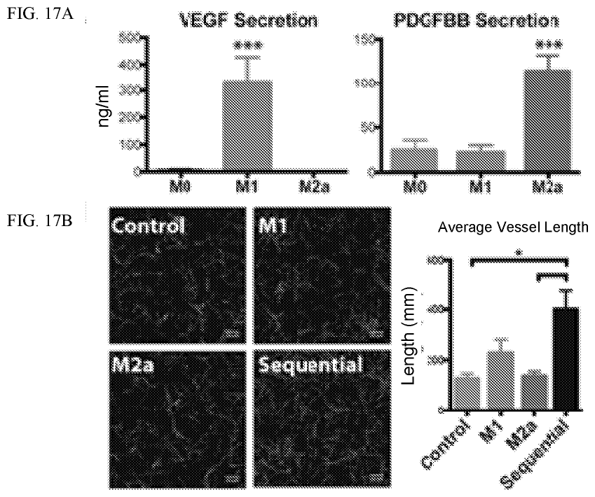

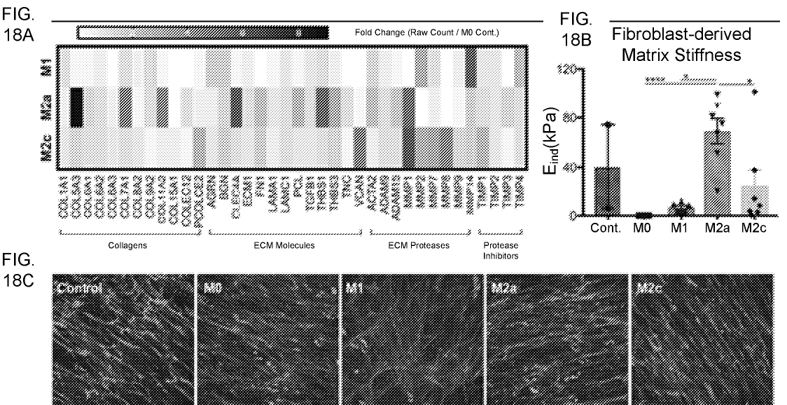

Macrophage genes in normal and chronic wound healing. Finally, a publicly available data set of human burn wound healing was used to investigate the timing of the M1, M2a, and M2c phenotypes using gene expression markers. The top 100 markers of each phenotype from the burn data set were clustered into genes with similar temporal trends (FIGS. 19A-19D). After interrogating the composition of each cluster, it was found that M1 and M2c genes were primarily associated with the early stages of healing (FIGS. 19A and 19C), while M2a genes were primarily associated with the later stages (FIGS. 19B and 19C). These findings are in agreement with studies using animal models that have tracked macrophages on the cellular level using markers that are known to be associated with each phenotype, supporting the use of gene expression markers to track phenotype. This analysis also allowed us to identify gene markers that are particularly important in the early and late stages of human wound healing. In a preliminary analysis of these genes in human DFU healing, it was found that patients whose wounds failed to heal in response to the standard of care exhibited a higher ratio of early stage to late stage genes (FIG. 19D).

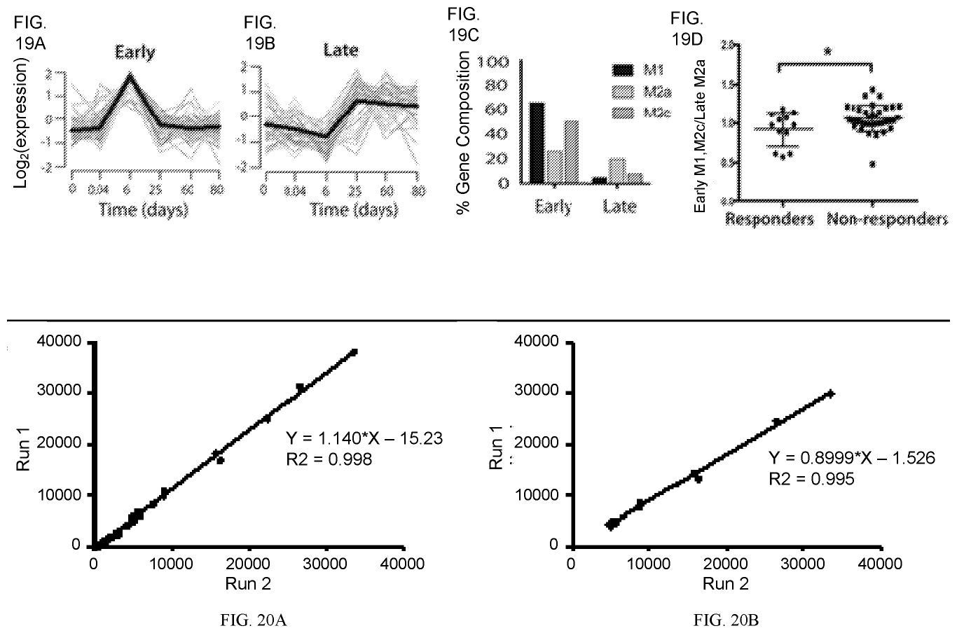

M1/M2a Score. Rather than trying to directly measure the numbers of M1 and M2a macrophages over time, which has limited translational potential for previously described reasons, these concepts were instead applied to discover a healing signature. Because of patient-to-patient variability and heterogeneous samples, the challenge was to develop a robust assay that would accurately predict healing across different patients and samples. Debrided wound tissue was collected from the DFUs of 10 patients over 4 weeks; treatment and follow-up were conducted for an additional 8 weeks to determine objectively if the ulcer had healed at 12 weeks. Interestingly, none of the 7 selected genes (IL1B, CCR7, CD80, VEGF, MRC1, TIMP3, PDGFB) were significantly differentially expressed between healing and non-healing DFUs. To normalize the data so that it was not affected by the number of macrophages contained within the sample while magnifying potential differences in macrophage phenotype, the data from all 7 genes were converted into a combinatorial M1/M2a ratio, so that it was higher for M1 macrophages and lower for M2a macrophages, which was validated using human macrophages that were prepared in vitro (FIG. 20A). A publicly available data set of human burn wounds was used to show that this M1/M2a score peaks immediately after injury, and then decreases over time, in agreement with studies that described the M1-to-M2a transition in normal wound healing (FIG. 20B). When applied to debrided DFU tissue collected over time, it was found that the M1/M2a scores decreased for DFUs that successfully healed by 12 weeks (FIG. 20C). In stark contrast, the scores stayed the same or increased for DFUs that ultimately failed to heal by 12 weeks. In fact, the fold change in the score at 4 weeks from the initial visit was almost 100 times higher for non-healing DFUs compared to healing DFUs (p<0.0001), and successfully predicted healing or non-healing in all 10 patients in this study (n=5 healing and n=5 non-healing; where a fold change at 4 weeks of less than 1 indicates healing and greater than 1 indicates non-healing). The results of this first study were published in Nassiri et al., Relative Expression of Proinflammatory and Antiinflammatory Genes Reveals Differences between Healing and Non-healing Human Chronic Diabetic Foot Ulcers, J Invest Dermatol. 2015; 135(6):1700-3. In a follow-up study, the same trends accurately predicted healing in all 6 patients in that study (n=4 healing and n=2 non-healing; published in Bajpai et al., Effects of nonthermal, noncavitational ultrasound exposure on human diabetic ulcer healing and inflammatory gene expression in a pilot study, Ultrasound in Medicine and Biology 2018; 44(9):2043-9). Finally, a third study showed that the method was accurate for 3 of 5 non-healing patients, for a total of 19 of 21 correct classifications (83% sensitivity and 100% specificity). These results indicate that the M1/M2a score may be useful as a surrogate biomarker of wound healing. However, as previously mentioned, it is important to note that the selected genes are not specific to macrophages, so it is likely that the M1/M2a score would be more aptly described as a marker of inflammation as opposed to a measure of macrophage phenotype per se.

Neural network. Neural networks are used to develop an assay that could use a single sample from the first visit to predict if patients are likely to respond to the standard of care (offloading, debridement, and simple moist wound dressings), so that they could be fast-tracked to more aggressive treatments if necessary. NANOSTRING® technology was used to analyze gene expression of a panel of 227 macrophage phenotype-related genes that previously identified to be differentially regulated over time in normal wound healing in debrided tissue samples collected at the first clinical visit from 13 patients with chronic DFUs. The top 10 most highly expressed genes (which were mostly associated with the M1 and M2c macrophage phenotypes) were used to build a neural network-style machine learning algorithm to classify healing outcome at 12 weeks as one of three possible outcomes: fully closed, remains open, or necessitates amputation (based on the decision of the treating clinician, who was blinded to the results of this study) (FIG. 21). As a preliminary step to determine the ideal number of hidden layers, or instances in which a linear function is applied to the inputs, 10-fold cross validation was performed for 1 through 10 hidden layers. The cross validation method involves splitting the data into unique sets and iterating through each point as a test set while the others are used for training. This strategy allows every sample to be used to test the model in order to improve its performance on new data with the same number of hidden units. The number of hidden layers that minimized the training error was 9. Finally, the resultant algorithm with 9 hidden layers was then tested on an additional 10 patients as a validation set. The algorithm correctly classified the 12-week outcome for 7 of the 10 patients (Table 2). These results suggest that analysis of a single sample at the start of treatment can be used to predict responsiveness to the standard of care.

TABLE 2
Classification accuracy of the neural network model.

| | Predicted | | |
|---|---|---|---|
| | Fully | | Necessitates |
| Actual | healed | Remains Open | Amputation |
| Fully healed | 4 | 1 | 0 |
| Remains | 1 | 1 | 0 |
| open | | | |
| Necessitates | 1 | 0 | 2 |
| amputation | | | |

Total accurate predictions: 7/10

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method of predicting whether a wound will heal or will not heal, the computer-implemented method comprising:
training a machine-learning algorithm utilizing at least:
gene-expression values for at least m genes from a first clinical encounter for each of a plurality of training subjects; and
a clinical diagnosis of a wound for each of the associated training subjects at a second, temporally later clinical encounter; and
applying the previously trained machine-learning algorithm to gene-expression values for a corresponding set of m genes from a new subject having a wound; and
presenting a prediction of whether the wound will heal generated by the previously trained artificial neural network machine-learning algorithm.

2. The method of claim 1, wherein the machine learning algorithm is an artificial neural network, a support vector machine, a binary classifier or series of binary classifiers or a decision tree.

3. The method according to claim 1, wherein m is selected from the group consisting of 10, 50, 100, 500 and 1000.

4. The method of claim 1, wherein the plurality of training subjects comprises:
a first subject group receiving a first wound treatment, and
a second plurality of subjects receiving a second wound treatment.

5. The method of claim 1, wherein:
the training step further utilizes gene expression values associated with the first and second wound treatment for the associated training subjects; and
the applying step further provides a candidate wound treatment as an input to the previously trained machine-learning algorithm.

6. The method of claim 5, wherein the method further comprises proposing an optimum wound treatment for the new subject based on the gene expression values from the new subject.

7. The method of claim 5, wherein the gene expression values are derived from a sample of debrided wound tissue.

8. The method of claim 7, wherein the sample of debrided wound tissue is collected at the first clinical encounter, stored in RNA-stabilizing solution and frozen until analysis.

9. The method of claim 5, wherein the gene expression values are derived by quantitative real-time polymerase chain reaction or by using a multiplex or high throughput gene expression analysis platform.

10. The method of claim 1, wherein the wound is a diabetic ulcer.

11. The method of claim 1, wherein the wound is a diabetic foot ulcer, a diabetic ulcer of the leg, a venous ulcer or a pressure ulcer.
